(19) United States
(12) Patent Application Publication
(10) Pub. No.: US 2007/0093511 A1
(43) Pub. Date: Apr. 26, 2007

(54) Anhydrous Crystal Form of Valaciclovir Hydrochloride

(76) Inventors: Helene S. Fain, Tonbridge (GB); David Alan Jones, Tonbridge (GB); Philip G. Lake, Dartford (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/552,218

(22) Filed: Oct. 24, 2006

Related U.S. Application Data
(63) Continuation of application No. PCT/US02/33926, filed on Oct. 23, 2002.
(60) Provisional application No. 60/333,313, filed on Nov. 5, 2001.

(51) Int. Cl. A61K 31/522; C07D 473/12

(52) U.S. Cl. 514/263.38; 544/276

(57) ABSTRACT

The invention relates to anhydrous crystalline valaciclovir hydrochloride, pharmaceutical compositions containing the same, its use in medical therapy and processes for preparing the same.

### BACKGROUND OF THE INVENTION

The present invention relates to a crystalline form of the antiviral compound valaciclovir hydrochloride, pharmaceutical formulations comprising this crystalline form, their use in therapy and processes for preparing the same.

The L-valine ester of acyclovir, namely (2-[2-amino-1,6-dihydro-6-oxo-purin-9-yl)methoxylethyl L-valinate, (otherwise known as valaciclovir) has been shown to possess much improved bioavailability while retaining the antiviral properties of acyclovir. A preferred form of this compound is its hydrochloride salt which is otherwise known as valaciclovir hydrochloride. The l-valinate ester of acyclovir and its salts including the hydrochloride salts are disclosed in U.S. Pat. No. 4,957,924, European patent no. 0308,065 and Beauchamp et al., Antiviral Chemistry and Chemotherapy, 3(3):157-164 (1992), the subject matter of which is incorporated herein by reference in their entirety.

An anhydrous crystal form of valaciclovir hydrochloride was found and characterized and is described in U.S. Pat. No. 6,107,302, the subject matter of which is incorporated herein by reference in its entirety. This crystal form is characterized by the X-ray powder diffraction pattern described in the '302 patent.

### BRIEF SUMMARY OF THE INVENTION

As a first aspect, the present invention provides anhydrous crystalline valaciclovir hydrochloride characterized by substantially the same infrared (IR) absorption spectrum as FIG. 1, wherein the IR absorption spectrum is obtained using a mull in mineral oil on an FT-IR spectrometer at 2 cm⁻¹ resolution.

As a second aspect, the present invention provides anhydrous crystalline valaciclovir hydrochloride characterized by an IR absorption spectrum obtained using a mull in mineral oil on an FT-IR spectrometer at 2 cm⁻¹ resolution, comprising peaks at five or more positions selected from the group consisting of 3286±1, 3197±1, 1750±1, 1686±1, 1632±1, 1607±1, 1152±1, 701±1, and 688±1 cm⁻¹.

As a third aspect, the present invention provides anhydrous crystalline valaciclovir hydrochloride characterized by substantially the same X-ray powder diffraction (XRD) pattern as FIG. 2, wherein the XRD pattern is expressed in terms of 2 theta angles and obtained with a diffractometer equipped with a diffracted beam graphite monochromator using copper kα X-radiation.

As a fourth aspect, the present invention provides anhydrous crystalline valaciclovir hydrochloride characterized by an XRD pattern expressed in terms of 2 theta angles and obtained with a diffractometer equipped with a diffracted beam graphite monochromator using copper Kα X-radiation, wherein the XRD pattern comprises 2 theta angles at four or more positions selected from the group consisting of 6.7±0.1, 8.1±0.1, 9.3±0.1, 11.4±0.1, 13.9±0.1, 15.7±0.1, 16.3±0.1, and 17.1±0.1 degrees.

As a fifth aspect, the present invention provides anhydrous crystalline valaciclovir hydrochloride characterized by an XRD pattern expressed in terms of 2 theta angles and obtained with a diffractometer equipped with a diffracted beam graphite monochromator using copper Kα X-radiation, wherein the XRD pattern comprises 2 theta angles 6.7±0.1, 8.1±0.1, 9.3±0.1, and 11.4±0.1 degrees.

As a sixth aspect, the present invention provides anhydrous crystalline valaciclovir hydrochloride characterized by substantially the same Raman spectrum as FIG. 3, wherein the Raman spectrum is obtained using a FT-Raman spectrometer at 4 cm⁻¹ resolution.

As a seventh aspect, the present invention provides anhydrous crystalline valaciclovir hydrochloride characterized by a Raman spectrum obtained using a FT-Raman spectrometer at 4 cm⁻¹ resolution, wherein the Raman spectrum comprises at least four peaks selected from the group consisting of 1684±1, 1364±1, 1348±1, 1191±1, and 810±1 cm⁻¹.

As an eighth aspect, the present invention provides anhydrous crystalline valaciclovir hydrochloride characterized by substantially the same solid state nuclear magnetic resonance (NMR) spectrum as FIG. 4, wherein the solid state NMR is obtained on a spectrometer operating at a frequency of 90.55 MHz for ¹³C observation at a temperature of 300K, a spinning speed 10 kHz and a recycle delay of 15 seconds.

As a ninth aspect, the present invention provides anhydrous crystalline valaciclovir hydrochloride characterized by a solid state NMR spectrum obtained using a spectrometer operating at a frequency of 90.55 MHz for ¹³C observation at a temperature of 300K, a spinning speed of 10 kHz and a recycle delay of 15 seconds, wherein the solid state NMR comprises chemical shifts at 15.1±0.1, 17.2±0.1, 20.2±0.1, 20.9±0.1, 29.2±0.1, 29.9±0.1, 58.4±0.1, 64.6±0.1, 66.8±0.1, 69.3±0.1, 70.7±0.1, 73.9±0.1, 74.4±0.1, 116.6±0.1, 117.3±0.1, 140.4±0.1, 150.4±0.1, 151.3±0.1, 153.6±0.1, 158.3±0.1, 169.1±0.1 and 169.6±0.1 ppm.

As another aspect, the present invention provides a pharmaceutical composition comprising anhydrous crystalline valaciclovir hydrochloride according to the present invention. The pharmaceutical composition may further comprise one or more pharmaceutically acceptable carriers or diluents.

As another aspect, the present invention provides a composition comprising anhydrous crystalline valaciclovir hydrochloride according to the present invention and hydrated valaciclovir hydrochloride.

As another aspect, the present invention provides a composition comprising anhydrous crystalline valaciclovir hydrochloride according to the present invention and Form 1 valaciclovir hydrochloride.

As another aspect, the present invention provides a method for the treatment or prophylaxis of a herpes viral infection in a mammal comprising administering to the mammal, an effective amount of anhydrous crystalline valaciclovir hydrochloride according to the present invention. The herpes viral infection may be selected from the group consisting of herpes simplex virus 1, herpes simplex virus 2, cytomegalovirs, Epstein Barr virus, varicella zoster virus, human herpes virus 6, human herpes virus 7,and human herpes virus 8.

As another aspect, the present invention provides a method for the treatment or prophylaxis of a condition or disease associated with a herpes viral infection in a mammal, comprising administering to the mammal an effective amount of anhydrous crystalline valaciclovir hydrochloride according to the present invention.

As another aspect, the present invention provides anhydrous crystalline valaciclovir hydrochloride according to the present invention for use in therapy.

As another aspect, the present invention provides the use of anhydrous crystalline valaciclovir hydrochloride according to the present invention in the preparation of a medicament for the treatment of prophylaxis of herpes viral infection.

As another aspect, the present invention provides the use of anhydrous crystalline valaciclovir hydrochloride according to the present invention in the preparation of a medicament for the treatment or prophylaxis of a condition or disease associated with a herpes viral infection.

As another aspect, the present invention provides a process for preparing anhydrous crystalline valaciclovir hydrochloride according to the present invention comprising slurrying damp valaciclovir hydrochloride or hydrated valaciclovir hydrochloride in a solvent capable of removing water by azeotropic distillation, under azeotropic distillation conditions.

As another aspect, the present invention provides another process for preparing anhydrous crystalline valaciclovir hydrochloride according to the present invention comprising the steps of:
a) optionally removing unbound process solvent from damp valaciclovir hydrochloride to provide hydrated valaciclovir hydrochloride;
b) slurrying damp valaciclovir hydrochloride or hydrated valaciclovir hydrochloride in a solvent capable of removing water by azeotropic distillation, under azeotropic distillation conditions to prepare the anhydrous crystalline valaciclovir hydrochloride; and
c) isolating the anhydrous crystalline valaciclovir hydrochloride.

As another aspect, the present invention provides a process for preparing anhydrous crystalline valaciclovir hydrochloride according to the present invention comprising the steps of:
a) removing unbound process solvent from damp valaciclovir hydrochloride to provide hydrated valaciclovir hydrochloride;
b) slurrying hydrated valaciclovir hydrochloride in an anhydrous solvent at a temperature of from about ambient temperature to about the boiling point of the anhydrous solvent for a period of time sufficient to convert the hydrated valaciclovir hydrochloride to the anhydrous crystalline valaciclovir hydrochloride; and
c) isolating the anhydrous crystalline valaciclovir hydrochloride.

### BRIEF DESCRIPTION OF THE SEVERAL VIEWS OF THE DRAWINGS

FIG. 1. The IR absorption spectrum of the anhydrous crystal form of valaciclovir hydrochloride according to the present invention (“Form 2 valaciclovir hydrochloride”). The x-axis is wavenumber in cm⁻¹ and the y-axis is percent transmittance. The IR absorption spectrum is obtained using a mull in mineral oil on an FT-IR spectrometer at 2 cm⁻¹ resolution according to the procedures described herein.

FIG. 2. The XRD pattern of Form 2 valaciclovir hydrochloride according to the present invention. The XRD pattern is expressed in terms of 2 theta angles and obtained with a diffractometer equipped with a diffracted beam graphite monochromator using copper Kα X-radiation, according to the procedures described herein.

FIG. 3. The Raman spectrum of Form 2 valaciclovir hydrochloride according to the present invention. The Raman spectrum is obtained using a FT-Raman spectrometer at 4 cm−1 resolution and 400 mW power, with a minimum of 600 scans accumulation, a InGaAs detector, and a CaF2 beamsplitter, according to the procedures described herein.

FIG. 4. The solid state NMR spectrum of Form 2 valaciclovir hydrochloride according to the present invention. The solid state NMR spectrum is obtained on a spectrometer operating at a frequency of 90.55 MHz for ¹³C observation at a temperature of 300K, a spinning speed of 10 kHz and a recycle delay of 15 seconds, according to the procedures described herein.

FIG. 5. The differential scanning calorimetry (DSC) thermogram for Form 2 valaciclovir hydrochloride according to the present invention. The DSC was carried out on a Perkin-Elmer Pyris-1 DCS system at a scan rate of 10° C. per minute, using a sample size of 2.789 mg, according to the procedures described herein.

FIG. 6. The thermogravimetric analysis (TGA) of Form 2 valaciclovir hydrochloride according to the present invention. The TGA was carried out on a Perkin-elmer Pyris-1 TGA system at a scan rate of 10° C. per minute, suing a sample size of 3.757 mg, according to the procedures described herein.

FIG. 7. The IR spectrum of valaciclovir hydrochloride according to U.S. Pat. No. 6,107,302 (“Form 1 valaciclovir hydrochloride”). The IR absorption spectrum is obtained using a mull in mineral oil on an FT-IR spectrometer at 2 cm⁻¹ resolution according to the procedures described in the Comparative Examples.

FIG. 8. The solid state NMR spectrum of Form 1 valaciclovir hydrochloride. The solid state NMR spectrum is obtained on a spectrometer operating at a frequency of 90.55 MHz for ¹³C observation at a temperature of 300K, a spinning speed of 10 kHz and a recycle delay of 15 seconds, according to the procedures described in the Comparative Example.

FIG. 9. The IR spectrum of hydrated valaciclovir hydrochloride. The IR absorption spectrum is obtained using a mull in mineral oil on an FT-IR spectrometer at 2 cm⁻¹ resolution according to the procedures described in the Comparative Example.

FIG. 10. The solid state NMR spectrum of hydrated valaciclovir hydrochloride. The solid state NMR spectrum is obtained on a spectrometer operating at a frequency of 90.55 MHz for ¹³ observation at a temperature of 300K, a spinning speed of 10 kHz and a recycle delay of 15 seconds, according to the procedures described in the Comparative Example.

### DETAILED DESCRIPTION OF THE INVENTION

The present invention provides a novel anhydrous crystalline form of valaciclovir hydrochloride exhibiting one or more advantageous pharmaceutical properties or other advantages over hydrated and other anhydrous crystal forms of valaciclovir hydrochloride. The anhydrous crystal form of the present invention possesses as one distinct advantage, that it can be prepared by processes which are simpler and more economical, particularly on a commercial scale, than other forms of valaciclovir hydrochloride. Unit operations such as filtration and drying add greatly to the cost of a pharmaceutical product on large scale production. The particles of the anhydrous crystal form of the present invention are more easily dried and filtered allowing downstream processing advantages and/or cost of goods advantages. The processes for the preparation of the anhydrous crystal form of the present invention also show a high degree of robustness, an advantage for a highly regulated compound. Batches of this crystalline form can, by the processes of this invention, be made consistently to a high crystal form purity, i.e., where the proportion of hydrated and other anhydrous crystalline forms of valaciclovir hydrochloride is limited (particularly less than 10%, more particularly less than 5% and still more particularly less than 3%). As another advantage, the anhydrous crystal form of the present invention is stable and essentially non-hygroscopic. It also has good storage properties and can be readily formulated into pharmaceutical compositions such as tablets and capsules.

As another one of its advantages, the anhydrous crystal form of the present invention is in the form a more powdery material than Form 1 valaciclovir hydrochloride. This advantage reduces or eliminates the need for a pre-grinding stage to reduce larger, harder pellets into finer, more powdery material for formulating. The various forms of valaciclovir hydrochloride may be characterized and differentiated using a number of conventional analytical techniques, including but not limited to X-ray powder diffraction (XRD) patterns, infrared (IR) spectra, Raman spectra, differential scanning calorimetry (DSC), thermogravimetric analysis (TGA) and solid state NMR.

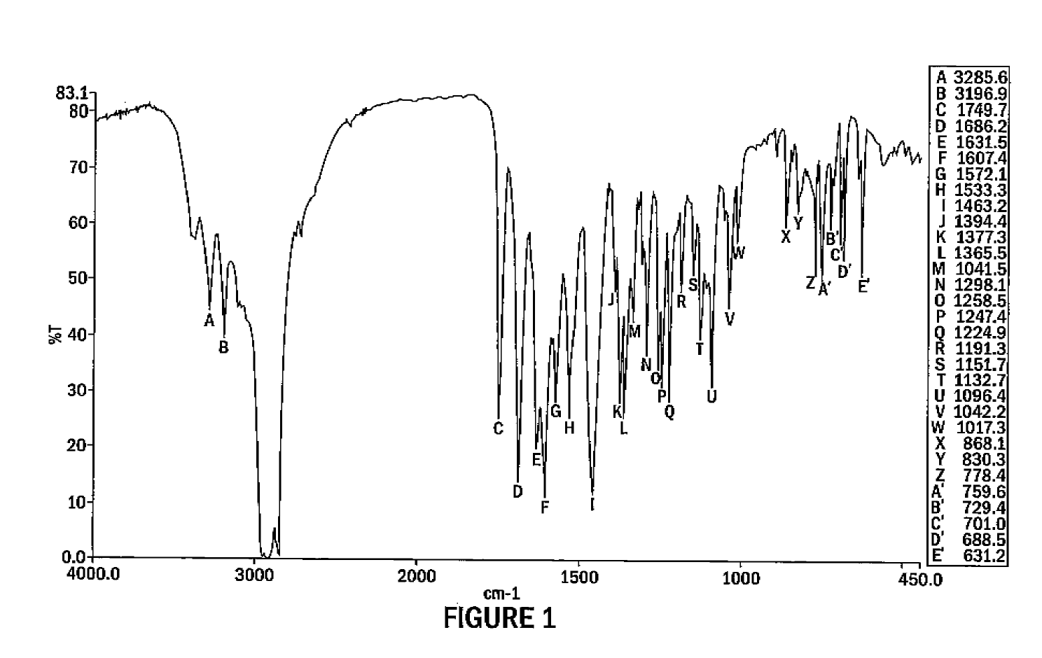

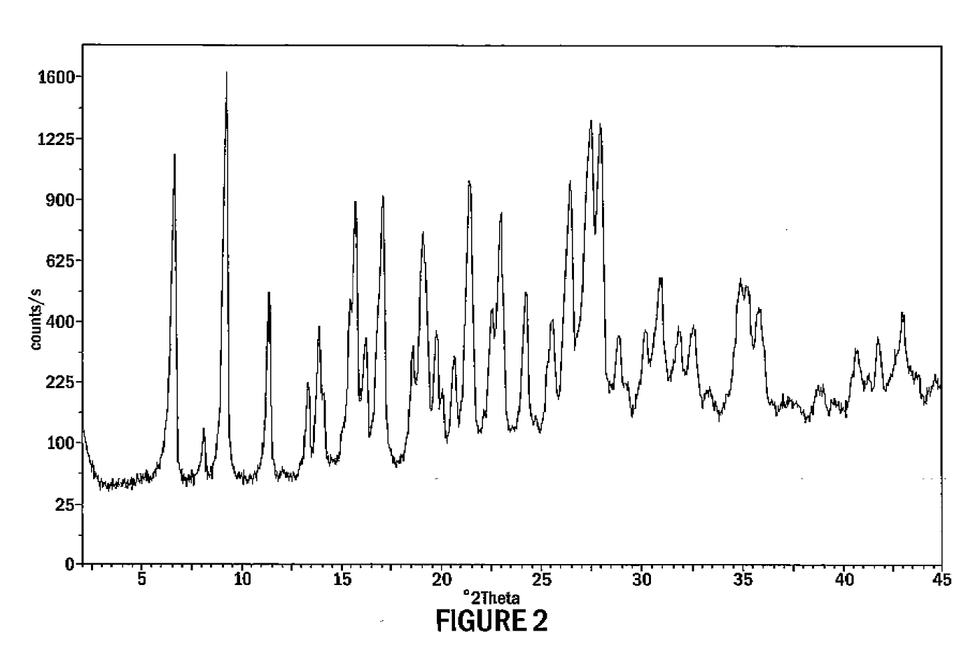

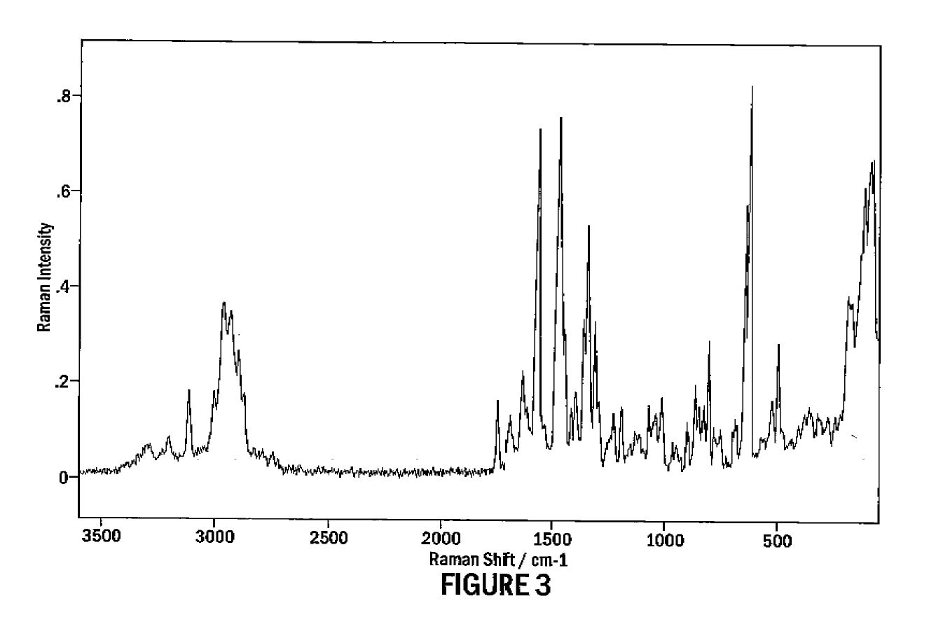

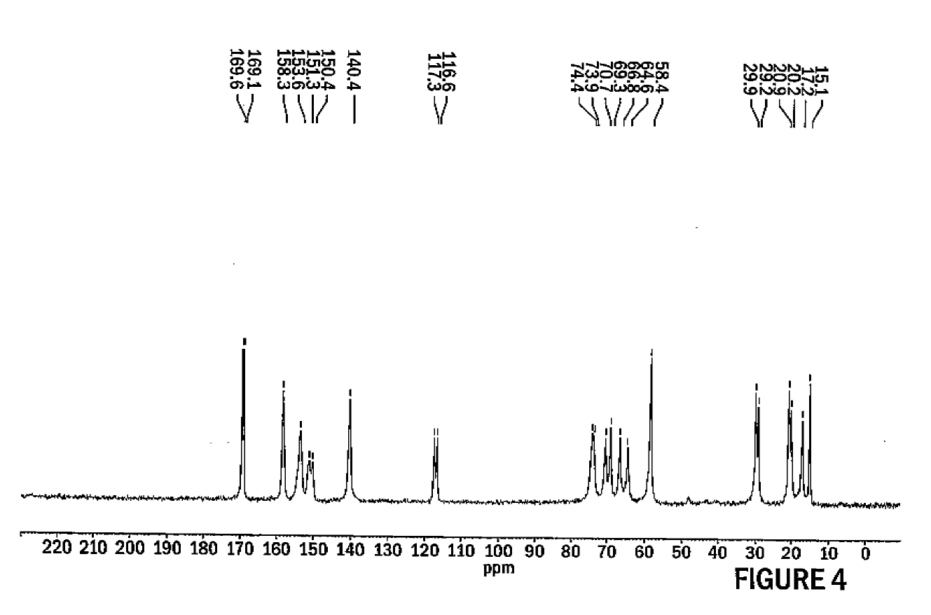

“Form 2 valaciclovir hydrochloride” as used herein refers to any of: 1) an anhydrous crystalline form of valaciclovir hydrochloride having substantially the same IR spectrum as shown in FIG. 1, obtained using a mull in mineral oil on an FT-IR spectrometer at 2 cm⁻¹ resolution; 2) an anhydrous crystalline form of valaciclovir hydrochloride having substantially the same XRD pattern as shown in FIG. 2 when measured with a properly aligned diffractometer equipped with a diffracted beam graphite monochromator using copper Kα X-radiation; 3) an anhydrous crystalline form of valaciclovir hydrochloride having substantially the same Raman spectrum as shown in FIG. 3, obtained using a FT-Raman spectrometer at 4 cm⁻¹ resolution; or 4) an anhydrous crystalline form of valaciclovir hydrochloride having substantially the same solid state NMR spectra a shown in FIG. 4, obtained on a spectrometer operating at a frequency of 90.55 MHz for ¹³C observation at a temperature of 300K, a spinning speed 10 kHz and a recycle delay of 15 seconds.

“Form 1 valaciclovir hydrochloride” as used herein shall refer to the anhydrous crystalline valaciclovir hydrochloride described in U.S. Pat. No. 6,107,302, having the identifying characteristics described therein.

“Hydrated valaciclovir hydrochloride” as used herein shall refer to any hydrated form of valaciclovir hydrochloride, including valaciclovir hydrochloride monohydrate, valaciclovir hydrochloride dihydrate and mixtures thereof.

“Damp valaciclovir hydrochloride” as used herein shall refer to the hydrated valaciclovir hydrochloride in the presence of process solvent.

“Process solvent” as used herein shall refer to any solvent employed for the preparation of valaciclovir hydrochloride, such as by recrystallization, by slurry, by the processes descried in either U.S. Pat. No. 4,957,924 or U.S. Pat. No. 6,107,302, or by any other suitable synthesis method.

The IR spectrum of the anhydrous crystalline form of valaciclovir hydrochloride according to the present invention (i.e., form 2 valaciclovir hydrochloride) can be determined using conventional equipment and techniques known to those skilled in the art of analytical chemistry and physical characterization. The IR spectra of FIGS. 1, 7, and 9 were obtained with a Perkin-Elmer System 2000 FT-IR spectrometer at 2 cm−1 resolution. The wavenumber in cm⁻¹ (x-axis) is plotted against percent transmittance (y-axis). All samples were prepared as a mull in mineral oil. Representative peaks observed in the IR spectrum of form 2 valaciclovir hydrochloride as a mull in mineral oil are as follows: 3286±1, 3197±1, 1750±1, 1686±1, 1632±1, 1607±1, 1572±1, 1533±1, 1463±1, 1394±1, 1377±1, 1365±1, 1341±1, 1298±1, 1258±1, 1247±1, 1224±1, 1191±1, 1152±1, 1132±1, 1096±1, 1042±1, 1017±1, 868±1, 830±1, 778±1, 759±1, 729±1, 701±1, 688±1 and 631±1 cm⁻¹.

As will be apparent to those skilled in the art, not all of these peaks are necessary to conclusively identify an analyzed sample as Form 2 valaciclovir hydrochloride, Form 2 valaciclovir hydrochloride can be identified by the presence of peaks at 5 or more positions selected from the group consisting of 3286±1, 3197±1, 1750±1, 1686±1, 1632±1, 1607±1, 1152±1, 701±1, and 688±1 cm⁻¹. More particularly, at least 7 of these peaks are present and in one embodiment, all of the foregoing peaks are present.

Slight variations in observed peaks are expected based on the specific spectrometer employed and the analyst's sample preparation technique. Some margin of error is present in each of the peak assignments reported above. The margin of error in the foregoing peak assignments is approximately ±1 cm⁻¹.

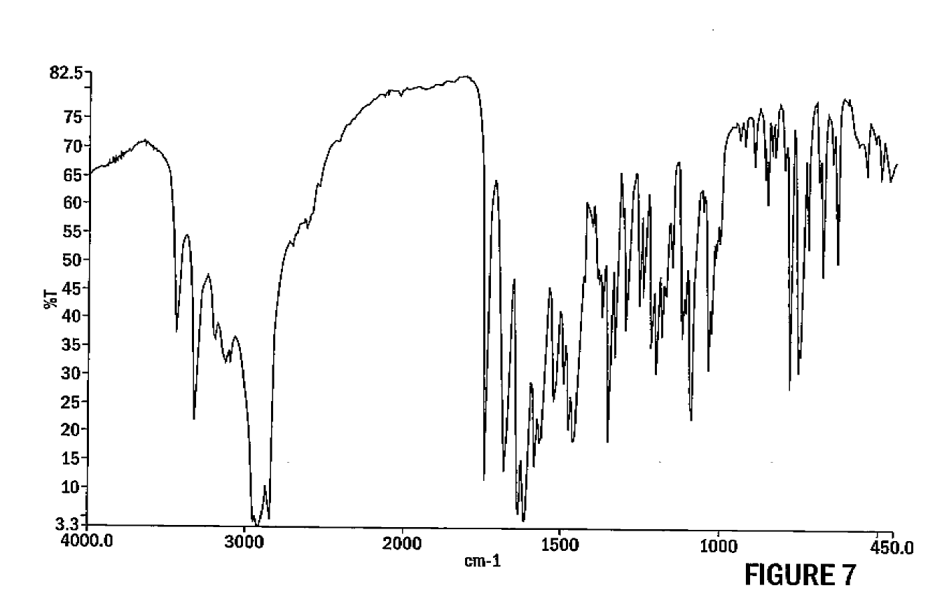

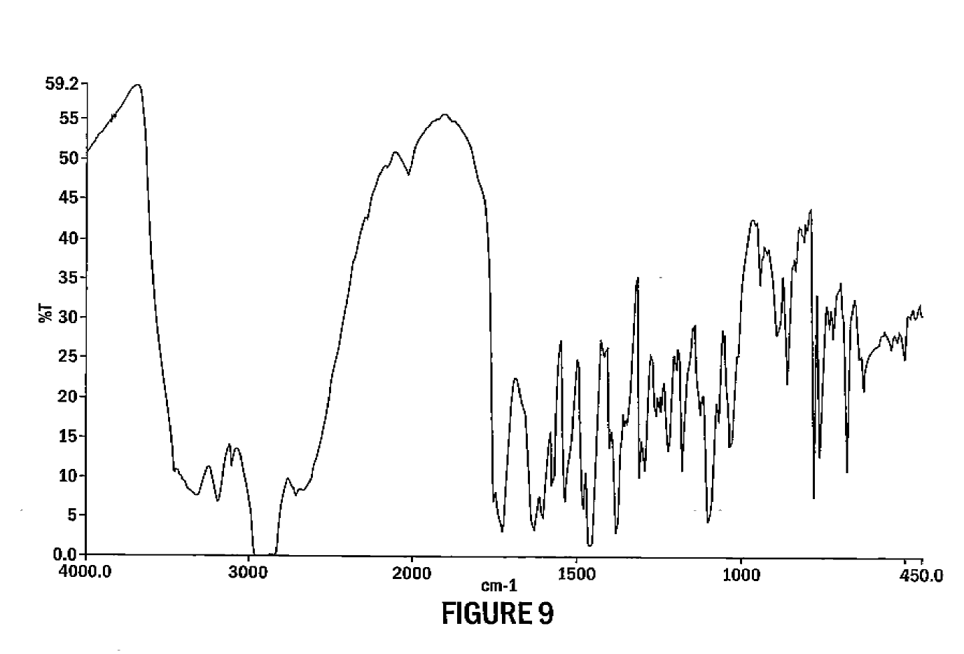

Since some margin of error is possible in the peak assignments, a useful method of comparing IR spectra in order to identify the particular form of a sample of valaciclovir hydrochloride is to overlay the IR spectrum of the sample over the IR spectrum of each of the known forms. For example, one skilled in the art can overlay an IR spectrum of an unknown form of valaciclovir hydrochloride, obtained using the methods described herein, over FIG. 1 and, using expertise and knowledge in the art, readily determine whether the IR spectrum of the unknown sample is substantially the same as the IR spectrum of Form 2 valaciclovir hydrochloride. If the IR spectrum is substantially the same as FIG. 1, the previously unknown form can be readily and accurately identified as Form 2 valaciclovir hydrochloride. FIGS. 7 and 9 can be used in the same manner to determine whether the sample is Form 1 valaciclovir hydrochloride or hydrated valaciclovir hydrochloride, respectively.

The X-ray powder diffraction pattern of Form 2 valaciclovir hydrochloride can be determined using conventional techniques and equipment known to those skilled in the art of analytical chemistry and physical characterization. The diffraction pattern of FIG. 2 was obtained with a Philips X-Pert Pro diffractometer system equipped with a diffracted beam graphite monochromator using copper Kα X-radiation and an automated divergent slit. A xenon proportional counter was used as the detector. The powder sample used to generate the X-ray powder diffraction data was prepared by conventional back filled sample preparation techniques using a 10 mm diameter holder about 1.5 mm thick.

A powder sample of Form 2 valaciclovir hydrochloride was used to produce the XRD pattern of FIG. 2. 2 Theta angles in degrees (x-axis) is plotted against peak intensity in terms of the count rate per seconds (y-axis). The XRD pattern for each anhydrous crystalline form and hydrated valaciclovir hydrochloride is unique to the particular form; exhibiting a unique set of diffraction peaks which can be expressed in 2 theta angles (°), d-spacings (Å) and/or relative peak intensities.

2 Theta diffraction angles and corresponding d-spacing values account for positions of various peaks in the XRD pattern. D-spacing values are calculated with observed 2 Theta angles and copper Kα1 wavelength using the Bragg equation. Slight variations in observed 2 theta angles and d-spacings are expected based on the specific diffractometer employed and the analyst's sample preparation technique. More variation is expected for the relative peak intensities. Identification of the exact crystal form of a compound should be based primarily on observed 2 theta angles or d-spacings with lesser importance place on relative peak intensities. To identify Form 2 valaciclovir hydrochloride, the certain characteristic 2 theta angle peaks occur at 6.7±0.1, 8.1±0.1, 9.3±0.1, and 11.4±0.1 degrees, or 24.63, 13.17, 10.88 and 9.52 Å d-spacing.

Although one skilled in the art can identify Form 2 valaciclovir hydrochloride from these characteristic 2 theta angle peaks, in some circumstances it may be desirable to rely upon additional 2 theta angles or d-spacings for the identification of Form 2 valaciclovir hydrochloride. In one embodiment at least five, particularly seven and more particularly all, of the following 2 theta angles are employed to identify Form 2 valaciclovir hydrochloride: 6.7±0.1, 8.1±0.1, 9.3±0.1, 11.4±0.1, 13.9±0.1, 15.7±0.1, 16.3±0.1, and 17.1±0.1 degrees.

Form 2 valaciclovir hydrochloride typically exhibits 2 theta angle peaks in addition to the foregoing peaks. For example, Form 2 valaciclovir hydrochloride may exhibit 2 theta angle peaks at essentially the following positions: 6.7±0.1, 8.1±0.1, 9.3±0.1, 11.4±0.1, 13.3±0.1, 13.9±0.1, 15.4±0.1, 15.7±0.1, 16.3±0.1, 17.1±0.1, 18.6±0.1, 19.1±0.1, 19.3±0.1, 19.8±0.1, 20.6±0.1, 21.4±0.1, 22.6±0.1, 22.9±0.1, 24.2±0.1, 25.5±0.1, 26.4±0.1, 27.2±0.1, 27.5±0.1, 27.8±0.1, 28.0±0.1, 28.9±0.1, 30.2±0.1, 30.9±0.1, 31.9±0.1, 32.6±0.1, 34.9±0.1, 35.3±0.1 and 35.9±0.1 degrees, or about 24.63, 13.17, 10.88, 9.52, 7.75, 7.27, 6.63, 6.36, 6.25, 6.07, 5.73, 5.62, 5.44, 5.18, 4.77, 4.67, 4.59, 4.49, 4.42, 4.30, 4.15, 3.94, 3.88, 3.67, 3.49, 3.38, 3.28, 3.24, 3.20, 3.18, 3.09, 3.04, 2.96, 2.89, 2.81, 2.74, 2.57, 2.54, 2.50, 2.43, 2.30, 2.21, 2.15 and 2.10 Å d-spacing. Some margin of error is present in each of the 2 theta angle assignments and d-spacings reported above. The error in determined d-spacings decreases with increasing diffraction scan angle or decreasing d-spacing. The margin of error in the foregoing 2 theta angles is approximately ±0.1 degrees for each of the foregoing peak assignments.

Since some margin of error is possible in the assignment of 2 theta angles and d-spacings, the preferred method of comparing XRD patterns in order to identify a the particular form of a sample of valaciclovir hydrochloride is to overlay the XRD pattern of the unknown sample over the XRD pattern of a known form. For example, one skilled in the art can overlay an XRD pattern of an unknown sample of valaciclovir hydrochloride, obtained using the methods described herein, over FIG. 2 and, using expertise and knowledge in the art, readily determine whether the XRD pattern of the unknown sample is substantially the same as the XRD pattern of Form 2 valaciclovir hydrochloride. If the XRD pattern is substantially the same as FIG. 2, the previously unknown form can be readily and accurately identified as Form 2 valaciclovir hydrochloride. The same technique can be used to determine if the unknown sample is Form 1 valaciclovir hydrochloride by overlaying the XRD pattern over FIGS. 1, 2 or 3 of U.S. Pat. No. 6,107,302.

Although 2 theta angles, or d-spacings are the primary method of identifying a particular crystalline form, it may be desirable to also compare relative peak intensities. As noted above, relative peak intensities may vary depending upon the specific diffractometer employed and the analyst's sample preparation technique. The peak intensities are reported as intensities relative to the peak intensity of the strongest peak. The intensity units on the XRD are counts/sec. The absolute counts=counts/time×count time=counts/sec×10 sec.

Considering 2 theta angles, d-spacing (Å) and relative peak intensity (I), Form 2 valaciclovir hydrochloride exhibits the following XRD pattern characteristics:

Form 2 Valaciclovir Hydrochloride

| 2 theta angle (°)¹ | Å | I |
|---|---|---|
| 3.6 | 24.63 | 0.2 |
| 6.7 | 13.17 | 75.6 |
| 8.1 | 10.88 | 5.2 |
| 9.3 | 9.52 | 100.0 |
| 11.4 | 7.75 | 28.3 |
| 12.2 | 7.27 | 2.5 |
| 13.3 | 6.63 | 11.2 |
| 13.9 | 6.36 | 16.9 |
| 14.2 | 6.25 | 8.7 |
| 14.6 | 6.07 | 7.0 |
| 15.5 | 5.73 | 22.1 |
| 15.8 | 5.62 | 40.8 |
| 16.3 | 5.44 | 18.5 |
| 17.1 | 5.18 | 48.4 |
| 18.6 | 4.77 | 13.6 |
| 19.0 | 4.67 | 26.5 |
| 19.3 | 4.59 | 17.1 |
| 19.8 | 4.49 | 16.1 |
| 20.1 | 4.42 | 9.8 |
| 20.6 | 4.30 | 11.3 |
| 21.4 | 4.15 | 50.0 |
| 22.6 | 3.94 | 18.9 |
| 22.9 | 3.88 | 34.1 |
| 24.2 | 3.67 | 23.7 |
| 25.5 | 3.49 | 18.8 |
| 26.4 | 3.38 | 49.7 |
| 27.2 | 3.28 | 32.0 |
| 27.5 | 3.24 | 50.9 |
| 27.8 | 3.20 | 46.9 |
| 28.0 | 3.18 | 52.0 |
| 28.9 | 3.09 | 14.9 |
| 29.3 | 3.04 | 6.4 |
| 30.2 | 2.96 | 11.8 |
| 31.0 | 2.89 | 18.8 |
| 31.9 | 2.81 | 13.5 |
| 32.7 | 2.74 | 12.0 |
| 34.9 | 2.57 | 14.4 |
| 35.3 | 2.54 | 14.5 |
| 35.9 | 2.50 | 10.3 |
| 37.0 | 2.43 | 3.2 |
| 39.1 | 2.30 | 3.2 |
| 40.7 | 2.21 | 5.2 |
| 41.9 | 2.15 | 4.8 |
| 43.1 | 2.10 | 7.3 |

¹Margin of error = approx. ±0.1 degrees.

Based upon the foregoing characteristic features of the XRD pattern of Form 2 valaciclovir hydrochloride, one skilled in the art can readily identify form 2 valaciclovir hydrochloride. It will be appreciated by those skilled in the art that the XRD pattern of a sample of Form 2 valaciclovir hydrochloride, obtained using the methods described herein, may exhibit additional peaks. The foregoing table provides the most intense peaks which are characteristic of that particular crystalline form or solvate. This table does not represent an exhaustive list of peaks exhibited by Form 2 valaciclovir hydrochloride.

Raman spectroscopy is another useful analytical technique for identifying the physical characteristics of a sample of valaciclovir hydrochloride and distinguishing between Form 2 valaciclovir hydrochloride, Form 1 valaciclovir hydrochloride and hydrated valaciclovir hydrochloride. The Raman spectrum of the anhydrous crystalline form of valaciclovir hydrochloride according to the present invention (i.e., Form 2 valaciclovir hydrochloride) can be determined using conventional equipment and techniques known to those skilled in the art of analytical chemistry and physical characterization. The Raman spectrum of FIG. 3 was obtained using a Nicolet 960 E.S.P. FT-Raman spectrometer. Data were acquired at 4 cm⁻¹ resolution. Laser excitation was at 1064 nm (as is inherent by the use of an FT-Raman spectrometer) with a power of 400 mW and a minimum of 600 scans accumulation. The number of sample scans was 1200 using an InGaAs detector and CaF₂ beamsplitter. Samples were prepared by placing the solid sample as received into a glass NMR tube. The sample was rotated during the measurement. In FIG. 3, Raman shift in cm⁻¹ (x-axis) is plotted against Raman intensity (y-axis).

The power (mW) and minimum number of scans accumulation may be adjusted within conventional knowledge to provide a spectrum of similar quality to that provided in FIG. 3. For example, if a higher power is employed, a lower number of minimum scans accumulation may be required to achieve a spectrum of similar quality to that reported in FIG. 3. Similarly, if a lower power is employed, a higher number of minimum scans accumulation may be required to obtain a spectrum of similar quality. Preferably, when determining whether the Raman spectrum of an unknown sample of valaciclovir hydrochloride is Form 2 valaciclovir hydrochloride, the spectrum will be obtained using a power of 400 mW and a minimum of 600 scans accumulation.

The choice of detector is not believed to be critical to obtaining a spectrum suitable for comparison with that provided at FIG. 3. As is known to those skilled in the art, a different detector will likely affect the intensity of the peaks. However, peak positions should remain relatively the same. For a definitive comparison, when determining whether the Raman spectrum of an unknown form of valaciclovir hydrochloride is Form 2 valaciclovir hydrochloride, preferably the spectrum will be obtained using an InGaAs detector.

Certain main peaks observed in the Raman spectrum of Form 2 valaciclovir hydrochloride as using an FT-Raman spectrometer at a resolution of 4 cm−1 are as follows: 3285±1, 3201±1, 3114.±1, 3003±1, 2960±1, 2931±1, 2894±1, 1749±1, 1684±1, 1630±1, 1568±1, 1477±1, 1449±1, 1416±1, 1397±1, 1364±1, 1348±1, 1310±1, 1226±1, 1191±1, 1133±1, 1070±1, 1039±1, 1014±1, 966±1, 902±1, 869±1, 850±1, 832±1, 810±1, 784±1, 760±1, 687±1, 646±1, 630±1, 527±1, 500±1, 364±1, 324±1, 278±1, 191±1, 120±1, 91±1 and 78±1 cm⁻¹.

As will be apparent to those skilled in the art, not all of these peaks are necessary to conclusively identify an analyzed sample as Form 2valaciclovir hydrochloride. Form 2 valaciclovir hydrochloride can be identified by the presence of peaks at 5 or more positions noted above. More particularly, at least 7 of these peaks are present and in one embodiment, all of the foregoing peaks are present. The most characteristic peaks of the Raman spectrum of Form 2 valaciclovir hydrochloride obtained using the foregoing methods, are at 1684±1, 1364±1, 1348±1, 1191±1, and 810±1 cm−1.

slight variations in observed peaks are expected based on the specific spectrometer employed, the resolution of the data and the analyst's sample preparation technique. Some margin of error is present in each of the peak assignments reported above. The margin of error in the foregoing peak assignments is approximately ±1 cm⁻¹.

Since some margin of error is possible in the peak assignments, the preferred method of determining whether an unknown form of valaciclovir hydrochloride is Form 2 valaciclovir hydrochloride is to overlay the Raman spectrum of the sample over the Raman spectrum provided in FIG. 3. One skilled in the art can overlay a Raman spectrum of an unknown form of valaciclovir hydrochloride, obtained using the methods described herein, over FIG. 3 and, using expertise and knowledge in the art, readily determine whether the Raman spectrum of the unknown sample is substantially the same as the Raman spectrum of Form 2 valaciclovir hydrochloride.

Solid state nuclear magnetic resonance (NMR) is yet another conventional analytical technique for identifying the physical characteristics of a sample of valaciclovir hydrochloride and distinguishing between Form 2 valaciclovir hydrochloride, Form 1 valaciclovir hydrochloride and hydrated valaciclovir hydrochloride. The solid state NMR spectrum of the anhydrous crystalline form of valaciclovir hydrochloride according to the present invention (i.e., Form 2 valaciclovir hydrochloride) is determined using conventional equipment and techniques known to those skilled in the art of analytical chemistry and physical characterization. The solid state NMR spectrum of FIGS. 4, 8 and 10 were obtained on a Bruker AMX360 spectrometer, operating at a frequency of 90.55 MHz for ¹³C observation at 300° K (i.e., ambient temperature) a spinning speed of 10 kHz and a recycle delay of 15 seconds. ¹³C MAS spectra are acquired by cross-polarisation from Hartmann-Hahn matched proton. 4 k data points were acquired in 60 ms, using a contact time of 3 ms and a recycle time of 15 s. Protons were decoupled during acquisition by using a two-pulse phase modulated (TPPM) composite sequence. The free induction decay (fid) was apodised by exponential multiplication using 5 Hz of line broadening before fourier transformation into 32 k data points. Chemical shifts were externally referenced to the carboxylate signal of glycine at 176.4 ppm relative to tetramethyl silane (TMS). Samples were prepared by placing the solid sample into a glass NMR tube. Chemical shift in ppm (x-axis) is plotted against intensity (y-axis).

Certain characteristic chemical shifts observed in the solid state NMR spectrum of Form 2 valaciclovir hydrochloride using a spectrometer operating at a frequency of 90.55 MHz for ¹³C observation at a temperature of 300K, a spinning speed 10 kHz and a recycle delay of 15 seconds include the following: 15.1±0.1, 17.2±0.1, 20.2±0.1, 20.9±0.1, 29.2±0.1, 29.9±0.1, 58.4±0.1, 64.6±0.1, 66.8±0.1, 69.3±0.1, 70.7±0.1, 73.9±0.1, 74.4±0.1, 116.6±0.1, 117.3±0.1, 140.4±0.1, 150.4±0.1, 151.3±0.1, 153.6±0.1, 158.3±0.1, 169.1±0.1 and 169.6±0.1 ppm

slight variations in observed chemical shifts are expected based on the specific spectrometer employed and the analyst's sample preparation technique. Some margin of error is present in each of the chemical shifts reported above. The margin of error in the foregoing chemical shifts is approximately ±0.1 ppm.

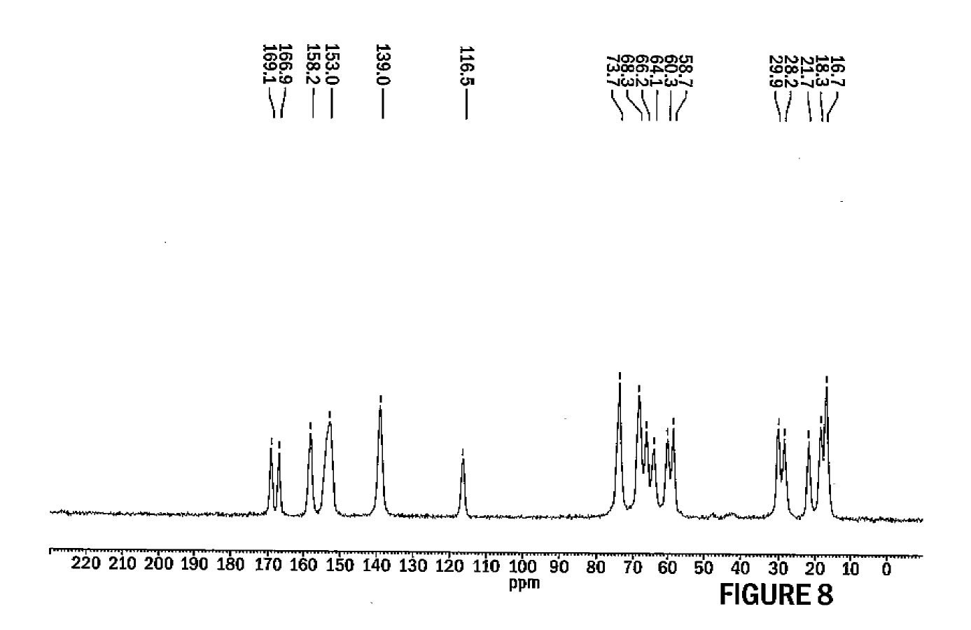

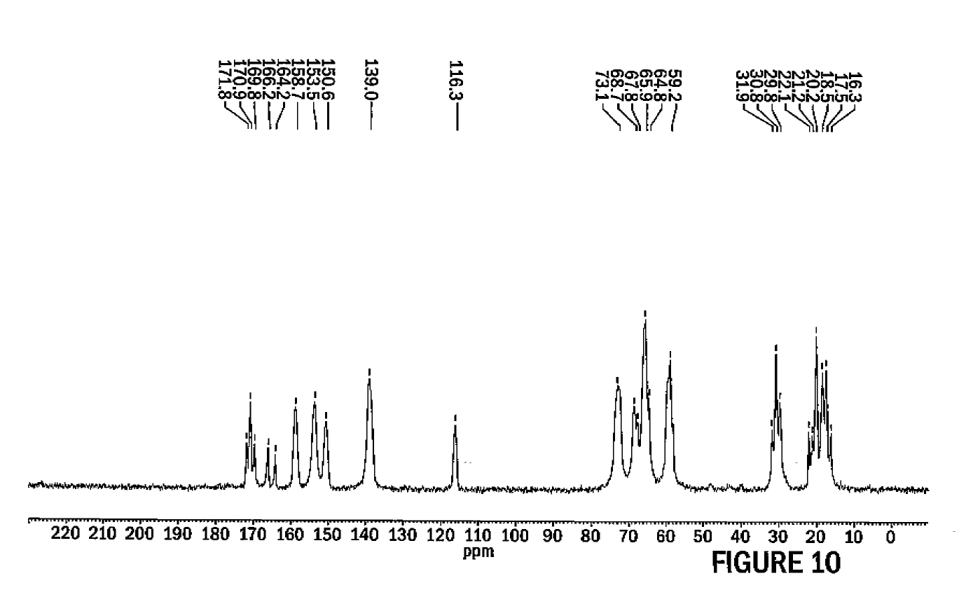

Since some margin of error is possible in the assignment of chemical shifts, the preferred method of determining whether an unknown form of valaciclovir hydrochloride is Form 2 valaciclovir hydrochloride is to overlay the solid state NMR spectrum of the sample over the solid state NMR spectrum provided in FIG. 4. One skilled in the art can overlay an NMR spectrum of an unknown sample of valaciclovir hydrochloride, obtained using the methods described herein, over FIG. 4 and, using expertise and knowledge in the art, readily determine whether the NMR spectrum of the unknown sample is substantially the same as the NMR spectrum of Form 2 valaciclovir hydrochloride. The same technique may be employed using FIGS. 8 and 10 to determine whether a particular sample is Form 1 valaciclovir hydrochloride or hydrated valaciclovir hydrochloride, respectively.

Any of the foregong analytical techniques can be used alone or in combination to identify a particular form of valaciclovir hydrochloride. In addition, other methods of physical characterization can also be employed to identify and characterize Form 2 valaciclovir hydrochloride. Exmaples of suitable techniques which are known to those skilled in the art to be useful for the physical characterization or identification of a crystalline form or solvate include but are not limited to melting point, differential scanning calorimetry, and thermogravimetric analysis. These techniques may be employed alone or in combination with other techniques to characterize a sample of an unknown form of valaciclovir hydrochloride, and to distinguish Form 2 valaciclovir hydrochloride from Form 1 and hydrated valaciclovir hydrochloride.

The present invention includes Form 2 valaciclovir hydrochloride both in substantially pure form and in admixture with other forms of valaciclovir hydrochloride; particularly one or both of hydrated valaciclovir hydrochloride and Form 1 valaciclovir hydrochloride. By “substantially pure” is meant that the composition comprises at least 90 percent Form 2 valaciclovir hydrochloride as compared to the other forms of valaciclovir hydrochloride in the composition, more particularly at least 95 percent Form 2 and in one embodiment, at least 94 percent Form 2 valaciclovir hydrochloride.

Form 2 valaciclovir hydrochloride may be in admixture with one or both of Form 1 valaciclovir hydrochloride or hydrated valaciclovir hydrochloride. Additionally, Form 2 may be in admixture with damp valaciclovir hydrochloride.

Since Form 2 valaciclovir hydrochloride is essentially free of water of hydration, the proportion of hydrated valaciclovir hydrochloride in any batch many be measured by the overall water of hydration content of each batch. In another aspect of the invention there is provided valaciclovir hydrochloride (either Form 2 valaciclovir hydrochloride or an admixture of Form 1 and Form 2 valaciclovir hydrochloride) having a water of hydration content of not more than 3% by weight (w/w) and including one or more of the characterizing data described above. More particularly, the water of hydration content is not more than 2% w/w, and in one embodiment, it is not more than 1.5% w/w and in still another embodiment, it is not more than 1% w/w and in yet another embodiment, it is not more than 0.7% w/w.

The water of hydration content is measured by the Karl Fischer method which is well known in the art and is described in the 1990 US Pharmacopoeia at pages 1619-1621, and the European Pharmacopoeia, second edition (1992) part 2, sixteenth fascicule at v. 3.5-6.1.

The present invention expressly contemplates the foregoing mixtures of Form 2 valaciclovir hydrochloride with one or more of Form 1 valaciclovir hydrochloride and hydrated valaciclovir hydrochloride. Admixtures of Form 2 valaciclovir hydrochloride with another form of the compound may result in the masking or absence of one or more of the foregoing X-ray powder diffraction peaks and Raman spectrum described above for Form 2 valaciclovir hydrochloride. Methods are known in the art for analyzing such admixtures of forms in order to provide for the accurate identification of the presence or absence of particular form in the admixture. Suitable methods for the quantitation of the particular forms in a mixture are well known in the art, e.g. IR, Raman, SSNMR, Near IR (NIR).

In another aspect, the present invention provides pharmaceutical compositions comprising Form 2 valaciclovir hydrochloride. Such pharmaceutical compositions may further comprise one or more other forms of valaciclovir hydrochloride and/or one or more pharmaceutically acceptable carriers or diluents. Examples of suitable pharmaceutical compositions and methods for their preparation are described in U.S. Pat. Nos. 4,957,924, 5,879,706 and PCT Publication No. WO01/82905, the subject matter of which is incorporated herein by reference in their entirety. Conveniently, suitable pharmaceutical compositions can be prepared using conventional techniques, and when employed, carriers and diluents. Pharmaceutical compositions for oral administration, such as tablet (and caplet) and capsule formulations, are preferred.

Form 2 valaciclovir hydrochloride for use in the instant invention may be used in combination with other therapeutic agents. Similarly, the pharmaceutical formulations of the present invention may include one or more additional therapeutic agents. Other therapeutic agents that may be combined with Form 2 valaciclovir hydrochloride include for example, non-nucleotide reverse transcriptase inhibitors, nucleoside reverse transcriptase inhibitors, protease inhibitors and/or other antiviral agents. The invention thus provides in a further aspect the use of a combination comprising Form 2 valaciclovir hydrochloride with a further therapeutic agent in the treatment of viral infections. Particular antiviral agents which may be combined with the compounds of the present invention include acyclovir, famcyclovir, gancyclovir, docosanol, miribavir, amprenavir, lamivudine, zidovudine, and abacavir.

When the compounds of formula (I) are used in combination with other therapeutic agents, the compounds may be administered either sequentially or simultaneously by any convenient route.

The combination referred to above may conveniently be presented for use in the form of a pharmaceutical formulation and thus pharmaceutical formulations, comprising a combination as defined above optionally together with a pharmaceutically acceptable carrier or diluent comprise a further aspect of the invention. The individual components of such combinations may be administered either sequentially or simultaneously in separate or combined pharmaceutical formulations.

When combined in the same formulation it will be appreciated that the two compounds must be stable and compatible with each other and the other components of the formulation and may be formulated for administration. When formulated separately they may be provided in any convenient formulation, in such a manner as is known for such compounds in the art.

When Form 2 valaciclovir hydrochloride is used in combination with a second therapeutic agent, the dose of each compound may differ from that when the compounds are used alone. Appropriate doses will be readily appreciated by those skilled in the art.

Form 2 valaciclovir hydrochloride and pharmaceutical compositions comprising the same are useful in therapy, particularly in the treatment or prophylaxis, including suppression of recurrence of symptoms, of a viral disease, in an animal, e.g. a mammal such as a human. The various therapeutic sues disclosed in U.S. Pat. Nos. 4,957,924, and 5,879,706 and PCT Publication no. WO 97/25989, the subject matter of which is incorporated herein by reference in their entirety, are similarly applicable to Form 2 valaciclovir hydrochloride. Form 2 valaciclovir hydrochloride is especially useful for the treatment or prophylaxis of viral diseases such as herpes viral infections. Herpes viral infections include, for example, herpes simplex virus 1 (HSV-1), herpes simplex virus 2 (HSV-2), cytomegalovirus (CMV) (including transplant CMV), Epstein Barr virus (EBV), varicella zoster virus (VZV) (also known as herpes zoster virus (HZV), human herpes virus 6 (HHV-6), human herpes virus 7 (HHV-7), and human herpes virus 8 (HHV-8). Form 2 valaciclovir hydrochloride is also useful in the treatment or prophylaxis of the symptoms or effects of herpes virus infections.

Form 2 valaciclovir hydrochloride is also useful in the treatment or prophylaxis of a condition or disease associated with a herpes virus infection, particularly a condition or disease associated with a latent herpes virus infection in an animal, e.g., a mammal such as a human. By “condition or disease associated with a herpes viral/virus infection” is meant a condition or disease, excluding the viral infection per se, which results from the presence of the viral infection, such as chronic fatigue syndrome which is associated with EBV infection and multiple sclerosis which has been associated with herpes viral infections such as EBV and HHV-6.

In addition to those conditions and diseases, Form 2 valaciclovir hydrochloride may also be sued for the treatment or prophylaxis of cardiovascular diseases and conditions associated with herpes virus infections, in particular atherosclerosis, coronary artery disease and restenosis and specifically restenosis following angioplasty (RFA). Restenosis is the narrowing of the blood vessels which can occur after injury to the vessel wall, for example injury caused by balloon angioplasty or other surgical and/or diagnostic techniques, and is characterized by excessive proliferation of smooth muscle cells in the walls of the blood vessel treated. It is thought that in many patients suffering from RFA, viral infection, particularly by CMV and/or HHV-6 of the patient plays a pivotal role in the proliferation of the smooth muscle cells in the coronary vessel treated. Restenosis can occur following a number of surgical and/or diagnostic techniques, for example, transplant surgery, vein grafting, coronary by-pass grafting and, most commonly following angioplasty.

There is evidence from work done both in vitro and in vivo, indicating that restenosis is a multifactorial process. Several cytokines and growth factors, acting in concert stimulate the migration and proliferation of vascular smooth muscle cells (SMC) and production of extracellular matrix material, which accumulate to occlude the blood vessel. In addition growth suppressors act to inhibit the proliferation of SMC's and production of extracellular matrix material.

The present invention provides a method for the treatment or prophylaxis of a viral infection in an animal such as a mammal (e.g., a human), particularly a herpes viral infection, which comprises administering to the animal an effective amount of Form 2 valaciclovir hydrochloride.

As used herein, the term “prophylaxis” refers to the prevention of infection, the prevention of occurrence of symptoms in an infected subject, or a decrease in severity or frequency of symptoms of viral infection, condition or disease in the subject.

As used herein, the term “treatment” refers to the partial or total elimination of symptoms or decrease in severity of symptoms of viral infection, condition or disease in the subject, or the elimination or decrease of viral presence in the subject.

As used herein, the term “effective amount” means an amount of a compound of formula (I) which is sufficient, in the subject to which it is administered, to treat or prevent the stated disease, condition or infection. For example, an effective amount of a compound of formula (I) for the treatment of a herpes virus infection is an amount sufficient to treat the herpes viral infection in the subject. The present invention also provides a method for the treatment or prophylaxis of a condition or disease associated with a herpes viral infection in an animal such as a mammal (e.g., a human), which comprises administering to the animal an effective amount of Form 2 valaciclovir hydrochloride. In one embodiment, the present invention provides a method for the treatment or prophylaxis of chronic fatigue syndrome or multiple sclerosis in an animal such as a mammal (e.g., a human), which comprises administering to the animal an effective amount of Form 2 valaciclovir hydrochloride. The foregoing method is particularly useful for the treatment or prophylaxis of chronic fatigue syndrome or multiple sclerosis, associated with latent infection with a herpes virus.

In another embodiment, the present invention provides a method for the treatment or prophylaxis of a cardiovascular condition such as atherosclerosis, coronary artery disease or restenosis (particularly restenosis following surgery such as angioplasty), which comprises administering to the animal an effective antiviral amount of Form 2 valaciclovir hydrochloride.

The present invention also provides the use of Form 2 valaciclovir hydrochloride in the preparation of a medicament for the treatment or prophylaxis of a viral infection in an animal such as a mammal (e.g., a human), particularly a herpes viral infection and the use of Form 2 valaciclovir hydrochloride in the preparation of a medicament for the treatment of a condition or disease associated with a herpes viral infection. In one embodiment, the present invention provides the use of a compound of formula (I) in the preparation of a medicament for the treatment or prophylaxis of cardiovascular disease, such as restenosis and atherosclerosis.

Simple dehydration of hydrated valaciclovir hydrochloride typically results in the formation of a partially amorphous and unstable form. The instantly claimed anhydrous crystal form can be conveniently prepared, however, by using the solvent mediated dehydrations described herein below. Accordingly, as a further aspect, the present invention provides a process for preparing Form 2 valaciclovir hydrochloride comprising slurrying damp valaciclovir hydrochloride or hydrated valaciclovir hydrochloride in a solvent capable of removing water by azeotropic distillation, under azeotropic distillation conditions. In one particular embodiment, the process comprises the steps of:
a) optionally removing unbound process solvent from damp valaciclovir hydrochloride to provide (substantially dry) hydrated valaciclovir hydrochloride;
b) slurrying the damp valaciclovir hydrochloride or the hydrated valaciclovir hydrochloride in a solvent capable of removing water by azeotropic distillation, under azeotropic distillation conditions to prepare said anhydrous crystalline valaciclovir hydrochloride; and
c) isolating the anhydrous crystalline (i.e., Form 2) valaciclovir hydrochloride.

Valaciclovir hydrochloride can be prepared using the processes described in U.S. Pat. No. 4,957,924 and 6,107,302, the subject matter of which is already incorporate herein by reference in their entirety. The synthesis of valaciclovir hydrochloride leads to the formation of hydrated valaciclovir hydrochloride in solution in the reaction mixture (i.e., in process solvent) form which it may be separated and purified as a solid product (i.e., damp valaciclovir hydrochloride).

Damp valaciclovir hydrochloride can be dried to remove unbound process solvent, thereby providing hydrated valaciclovir hydrochloride in substantially dry form. Drying can be accomplished by any suitable method. Examples of such methods are described in U.S. Pat. No. 6,107,302. In one preferred embodiment, unbound process solvent is removed from damp valaciclovir hydrochloride by slurrying damp valaciclovir hydrochloride in acetone, filtering and then drying, for example at about 30-70° C. To provide hydrated valaciclovir hydrochloride in substantially dry form.

Damp valaciclovir hydrochloride or hydrated valaciclovir hydrochloride may be used to prepare the anhydrous crystal form of the present invention. Certain factors influence which anhydrous crystal form results. These factors include, but are not limited to nucleation, seeding (both active and inadvertant), solvent mediated effects and critically water content. The solvent composition and solvent to product ratio is critical for the nucleation of the desired form. Typically seeding can influence the nucleation of the desired form from the solvent mixture. Variation in total water content of the processing solvent can also give rise to unexpected effects. In the following methods, conditions of separation and further processing are selected to produce the anhydrous crystalline form of the present invention (i.e., Form 2 valaciclovir hydrochloride).

According to the present method, either damp valaciclovir hydrochloride or hydrated valaciclovir hydrochloride is slurried in a solvent capable of removing water by azeotropic distillation. Suitable solvents capable of removing water by azeotropic distillation include but are not limited to C₁₋₆alcohols, ketones (such as C₁₋₆ ketones), esters (such as C₁₋₆ esters), ethers (such as C₁₋₆ ethers) and mixtures thereof. Specific examples of suitable solvents include but are not limited to butanol (e.g., butan-1-ol or butan-2-ol), propanol (e.g., propan-2-ol or propan-1-ol), toluene, ethyl acetate, butyl acetate, methyl isobutyl ketone and mixtures thereof. Additional solvents capable of removing water by azeotropic distillation which may be used in the processes of the present invention can be readily determined by those skilled in the art. Preferably, the solvent capable of removing water by azeotropic distillation is selected from the group consisting of butanol (e.g., butan-1-ol), ethyl acetate, methyl isobutyl ketone and mixtures thereof. In one preferred embodiment, the solvent capable of removing water by azeotropic distillation is butan-1-ol. In another preferred embodiment, the solvent capable of removing water by azeotropic distillation is methyl isobutyl ketone.

The step of slurrying the damp valaciclovir hydrochloride or hydrated valaciclovir hydrochloride in the above-described solvent is carried out by creating a thin, suspension of valaciclovir hydrochloride in the solvent, preferably with agitation.

The slurrying step takes place under azeotropic distillation conditions. Suitable azeotropic distillation conditions will be readily apparent to those skilled in the art and will depend upon the particular solvent selected. Typically, azeotropic distillation conditions involve heating the slurry, preferably with agitation, to the boiling point of the solvent capable of removing water by azeotropic distillation. The reaction is continued for a period of time sufficient to separate the water from the starting material, thus resulting in the anhydrous crystalline valaciclovir hydrochloride of the present invention. The amount of time required to convert to Form 2 valaciclovir hydrochloride will vary depending upon the particular solvent or mixture of solvents chosen, but typically, the reaction is carried out for from about 1 to about 6 hours.

the anhydrous crystalline valaciclovir hydrochloride produced by the slurrying process (i.e., Form 2 valaciclovir hydrochloride) may be isolated from the slurry by filtration.

Optionally, the process further comprises the additional step of drying the Form 2 valaciclovir hydrochloride. Drying may be accomplished in an oven at elevated temperature, with or without the presence of a desiccant, or at ambient temperature in the presence of a desiccant. In one embodiment, the product is dried under vacuum.

In another aspect, the present invention provides another process for preparing Form 2 valaciclovir hydrochloride comprising the steps of:
a) removing unbound process solvent from damp valaciclovir hydrochloride to provide (substantially dry) hydrated valaciclovir hydrochloride;
b) slurrying the hydrated valaciclovir hydrochloride in an anhydrous solvent at a temperature of from about ambient temperature to about the boiling point of the anhydrous solvent for a period of time sufficient to convert the hydrated valaciclovir hydrochloride to anhydrous crystalline valaciclovir hydrochloride according to the present invention; and
c) isolating the anhydrous crystalline valaciclovir hydrochloride.

The step of removing unbound process solvent from damp valaciclovir hydrochloride is described above.

The step of slurrying hydrated valaciclovir hydrochloride in an anhydrous solvent is carried out by creating a thin, suspension of hydrated valaciclovir hydrochloride in the solvent, preferably with agitation.

Suitable anhydrous solvents for use in the process of the present invention include but are not limited to water-free IMS, methanol, absolute ethanol, toluene, tetrahydrofuran, MIBK and mixtures thereof. Other suitable anhydrous solvents can be determined by those skilled in the art. In one embodiment, the anhydrous solvent is water-free IMS or absolute ethanol. In one embodiment, the anhydrous solvent is absolute ethanol, particularly absolute ethanol containing 2% or less water.

The slurrying step may be carried out at temperatures ranging from about ambient temperature up to the boiling point of the anhydrous solvent. According to this process, the temperature may be up to but not including the boiling point of the solvent; i.e., the temperature is not sufficiently high to boil the anhydrous solvent. Thus the temperature is lower than the boiling point of the anhydrous solvent. The optimum temperature for the slurrying step will depend upon the particular anhydrous solvent employed. Preferably the slurrying step is carried out at a temperature of from about 50 to about 60° C.

The slurrying step is carried out for a period of time sufficient to convert hydrated valaciclovir hydrochloride to Form 2 valaciclovir hydrochloride. The amount of time required to convert the hydrated valaciclovir hydrochloride to Form 2 valaciclovir hydrochloride will depend upon the choice of anhydrous solvent and the temperature at which the slurrying step is carried out. Typically, the slurrying step is carried out for from about 1 to about 24 hours, more aprticularly from about 1 to about 8 hours and in one embodiment from about 1 to about 2 hours.

The anhydrous crystalline valaciclovir (Form 2 valaciclovir hydrochloride) may be isolated by filtration. Optionally, the Form 2 valaciclovir hydrochloride thus produced may be dried as described above.

The following examples are intended for illustration only and are not intended to limit the scope of the invention in any way.

### EXAMPLE 1

### Preparation of Hydrated Valaciclovir Hydrochloride

Water (35 ml) was added to Form 1 valaciclovir hydrochloride (15 g). The mixture was heated at 60° C. with stirring until all the solids dissolved. Ethanol (70 ml) was added and the solution was allowed to cool to ambient temperature, the product started to precipitant after a few minutes. The mixture was cooled to 0-5° C. for 1 hour. The solid was collected by filtration, washed with ethanol (50 ml) and dried overnight under house vacuum to afford hydrated valaciclovir hydrochloride.

### EXAMPLE 2

### Preparation of Form 2 Valaciclovir Hydrochloride

A suspension of hydrated valaciclovir hydrochloride (Example 1) in butan-1-ol (100 ml) was heated at reflux and approximately 50 ml solvent removed by distillation. The suspension was stirred and heated at reflux for 1 hr then cooled to ambient temperature. Form 2 valaciclovir hydrochloride anhydrate was collected by filtration and dried in vacuo. Infra red analysis of the damp paste showed Form 2 valaciclovir hydrochloride.

### EXAMPLE 3

### Preparation of Form 2 Valaciclovir Hydrochloride

Butan-1-ol (50 ml) was added to hydrated valaciclovir hydrochloride (3.5 g). The suspension was heated and stirred at reflux. Approximately 20 ml solvent was removed by distillation and abutan-1-ol (50 ml) added. A further 30 ml solvent was removed by distillation followed by the addition of butan-1-ol (30 ml). The suspension was stirred and heated at reflux for 2.5 hrs. Form 2 valaciclovir hydrochloride was collected by filtration and dried in vacuo (2.64 g).

### EXAMPLE 4

### Preparation of Form 2 Valaciclovir Hydrochloride

4-Methyl 2-pentanone (30 ml) was added to hydrated valaciclovir hydrochloride (2 g), (Example 1). The suspension was stirred and heated in an oil bath at 120-130° C. for one hour. The temperature of the oil bath was increased to 150° C. and 15 ml of solvent was removed by azeotropic distillation, and the suspension was stirred for one further hour at an oil bath temperature of 120-130° C. The solid was collected by filtration and the reaction flask rinsed with 4-methyl-2-pentanone (10 ml). The solid was dried under vacuum over phosphorus pentoxide overnight to afford Form 2 valaciclovir hydrochloride.

### EXAMPLE 5

### Preparation of Form 2 Valaciclovir Hydrochloride

Industrial methylated spirit (IMS) (20 ml) was added to hydrated valaciclovir hydrochloride (2.0 g) (Example 1). The suspension was stirred at 50-60° C., for one hour. The solid was collected by filtration and the reaction flask rinsed with IMS (30 ml). The solid was dried under vacuum over phosphorus pentoxide overnight to afford Form 2 valaciclovir hydrochloride.

### EXAMPLE 6

### Preparation of Form 2 Valaciclovir Hydrochloride

Absolute ethanol (35 ml) was added to hydrated valaciclovir hydrochloride (2.0 g) (Example 1). The suspension was stirred at 50-60° C. for two hours. The solid was collected by filtration and the reaction flask rinsed with absolute ehtanol (30 ml). The solid was dried under vacuum over phosphorus pentoxide for two days to afford Form 2 valaciclovir hydrochloride.

### EXAMPLE 7

### Preparation of Form 2 Valaciclovir Hydrochloride

Absolute Ethanol (20 ml) was added to valaciclovir hydrochloride (2.0 g) (Example 1). The suspension was stirred at ambient temperature for four hours. The solid was collected by filtration and the reaction flask rinsed with absolute ethanol (30 ml). The solid was dried under vacuum over phosphorus pentoxide overnight to afford Form 2 valaciclovir hydrochloride.

### EXAMPLE 8

### Preparation of Form 2 Valaciclovir Hydrochloride

Absolute Ethanol (300 ml) was added to hydrated valacyclovir hydrochloride (30.0 g). The suspension was stirred at 50-60° C. for 1 hour. Absolute Ethanol (40 ml) was added and stirring at 50-60° C. was continued for 2.5 hours. The solid was collected by filtration and the reaction flask rinsed with aboslute ethanol (2×30 ml). The solid was dried under house vacuum over phosphorus pentoxide for three days. Then in vacuum oven for three hours to afford Form 2 valacyclovir hydrochloride (27.1 g).

### EXAMPLE 9

### Preparation of Form 2 Valaciclovir Hydrochloride

Tetrahydrofuran (20 ml) and methanol (20 ml) were added to hydrated valaciclovir hydrochloride (2.0 g) prepared in a similar fashion to that in Example 1. The suspension was stirred at ambient temperature for three and half hours. The solvents were evaporated under reduced pressure. The solid was dried under house vacuum on phosphorus pentoxide overnight to afford Form 2 valaciclovir hydrochloride.

### EXAMPLE 10

### Preparation of Form 2 Valaciclovir Hydrochloride

Ethyl acetate (50 ml) was added to hydrated valaciclovir hydrochloride (2.0 g). The suspension was heated and stirred at reflux. Approx. 30 ml solvent was added and removed by distillation. The suspension was stirred and heated at reflux for 3 hrs using a Dean and Stark apparatus. The suspension was cooled. Form 2 valaciclovir hydrochloride (1.08 g) was collected by filtration and dried in vacuo.

### EXAMPLE 11

### Analysis of Form 2 Valaciclovir Hydrochloride

Proton NMR.

The proton NMR spectrum was consistent with that of valaciclovir hydrochloride.

Water content (by Karl Fisher titration): 0.61% w/w

Infra Red.

The IR absorption spectrum of a mineral oil dispersion of the product was obtained using a Perkin-Elmer System 2000 FT-IR spectrometer at 2 cm⁻¹ resolution. Data were digitized at 0.5 cm⁻¹ intervals (FIG. 1). Bands were observed at (cm⁻¹): 3286, 3197, 1750, 1686, 1632, 1607, 1572, 1533, 1463, 1394, 1377, 1366, 1342, 1298, 1259, 1247, 1225, 1191, 1152, 1133, 1096, 1042, 1017, 868, 830, 778, 760, 729, 701, 689, 631, 570.

X-ray Powder Diffraction.

The XRD pattern was determined on a Philips X'Pert MPD diffractometer equipped with a monomchromator using copper Ka X-radiation. The Pattern is provided in FIG. 2. Characteristic XRD angles °20 (relative intensities %) 6.7 (75.63) 9.3 (100.0), 11.4 (28.34), 13.3 (11.23), 13.9 (16.91), 15.4 (22.07), 15.7 (40.81), 16.3 (18.54), 17.1 (48.40), 18.6 (13.55), 19.0 (26.45), 19.3 (17.11), 19.8 (16.07), 20.6 (11.32), 21.4 (50.03), 22.6 (18.93), 22.9 (34.14), 24.2 (23.67), 25.5 (18.76), 26.4 (49.69), 27.2 (31.95), 27.5 (50.86), 27.8 (46.94), 28.0 (51.96), 28.9 (14.85), 30.2 (11.80), 30.9 (18.75), 31.9 (13.47), 32.6 (11.99), 34.9 (14.40), 35.3 (14.54), 35.9 (10.28).

Raman.

Raman Spectra were collected on a Nicolet FT-Raman 960 running at 4 cm⁻¹ resolution and 400 mW power, with a minimum of 600 scans accumulation. Number of sample scans was 1200 using an InGaAs detector and CaF₂ beamsplitter. Spectrum is provided at FIG. 3. Shift bands were observed at (cm⁻¹); 3285, 3201, 3114, 3003, 2960, 2931, 2894, 1749, 1684, 1630, 1568, 1477, 1449, 1416, 1397, 1364, 1348, 1310, 1226, 1191, 1133, 1070, 1039, 1014, 966, 902, 869, 850, 832, 810, 784, 760, 687, 646, 630, 527, 500, 364, 324, 278, 191, 120, 91 and 78.

Thermal Analysis.

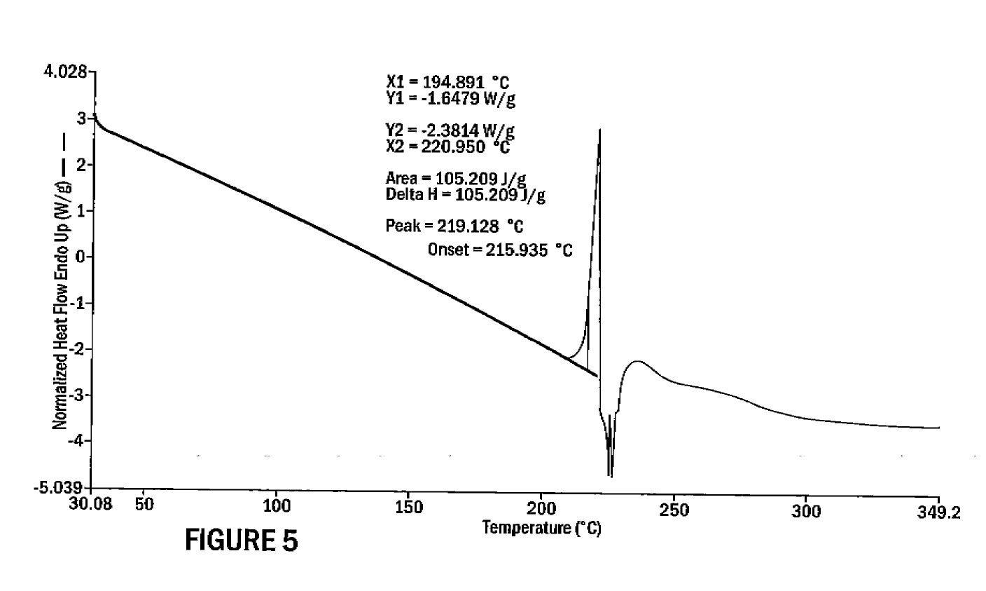

Differential scanning calorimetry was carried out on a Perkin-Elmer Pyris-1 DSC system. Scan rate of 10° C. per minute. Sample size 2.789 mg. The thermogram is provided at FIG. 5.

Moderately sharp asymmetric melting endotherm T=216° C. and exothermic decomposition is observed.

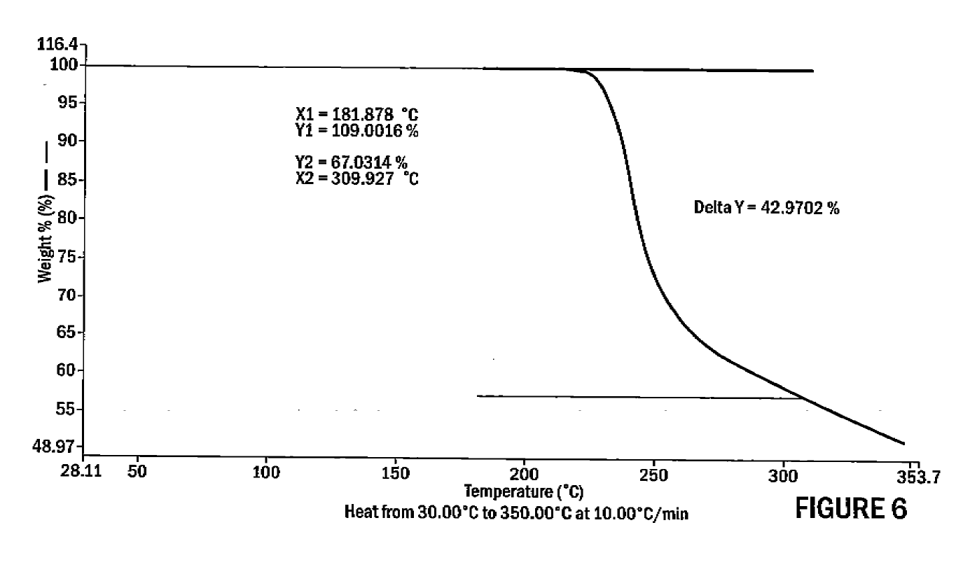

Thermogravimetric analysis was carried out on a Perkin-Elmer Pyris-1 TGA system. Scan rate of 10° C. per minute. Sample size 3.757 mg. The TGA trace is provided at FIG. 6.

Weight loss from 182-310° C.=42.97% w/w associated with melting/decomposition

Solid State Nuclear Magnetic Resonance

Acquisition was performed at 300K on a Bruker AMX360 spectrometer, operating at a frequency of 90.55 MHz for ¹³C observation. ¹³C MAS spectra are acquired by cross-polarization from Hartmann-Hahn matched proton. 4k data points were acquired in 60 ms, using a contact time of 3 ms and a recycle time of 15 s. Protons were decoupled during acquisition by using a two-pulse phase modulated (TPPM) composite sequence. The free induction decay (fid) was apodised by exponential multiplication using 5 Hz of line broadening before fourier transformation into 32 k data points. Chemical shifts were externally referenced to the carboxylate signal of glycine at 176.4 ppm relative to TMS. Spectrum is provided at FIG. 4.

### COMPARATIVE EXAMPLE

The IR and solid state NMR spectra of Form 1 valaciclovir hydrochloride (FIGS. 7 and 8, respectively) and hydrated valaciclovir hydrochloride (FIGS. 9 and 10, respectively) were obtained using procedures analogous to those described above in Example 11 for the IR and solid state NMR analysis of Form 2 valaciclovir hydrochloride.

The foregoing Examples are illustrative of the present invention and are not to be construed as limiting thereof. The invention is defined by the following claims including equivalents thereof.

1. Anhydrous crystalline valaciclovir hydrochloride characterized by substantially the same infrared (IR) absorption spectrum as FIG. 1, wherein said IR absorption spectrum is obtained using a mull in mineral oil on an FT-IR spectrometer at 2 cm⁻¹ resolution.

2. Anhydrous crystalline valaciclovir hydrochloride characterized by an IR absorption spectrum obtained using a mull in mineral oil on an FT-IR spectrometer at 2 cm⁻¹ resolution, comprising peaks at five or more positions selected from the group consisting of 3286±1, 3197±1, 1750±1, 1686±1, 1632±1, 1607±1, 1152±1, 701±1, and 688±1 cm⁻¹.

3. Anhydrous crystalline valaciclovir hydrochloride characterized by substantially the same X-ray powder diffraction (XRD) pattern as FIG. 2, wherein said XRD pattern is expressed in terms of 2 theta angles and obtained with a diffractometer equipped with a diffracted beam graphite monochromator using copper Kα X-radiation.

4. Anhydrous crystalline valaciclovir hydrochloride characterized by an XRD pattern expressed in terms of 2 theta angles and obtained with a diffractometer equipped with a diffracted beam graphite monochromator using copper Kα X-radiation, wherein said XRD pattern comprises 2 theta angles at four or more positions selected from the group consisting of 6.7±0.1, 8.1±0.1, 9.3±0.1, 11.4±0.1, 13.9±0.1, 15.7±0.1, 16.3±0.1, and 17.1±0.1 degrees.

5. Anhydrous crystalline valaciclovir hydrochloride characterized by an XRD pattern expressed in terms of 2 theta angles and obtained with a diffractometer equipped with a diffracted beam graphite monochromator using copper Kα X-radiation, wherein said XRD pattern comprises 2 theta angles at 6.7±0.1, 8.1±0.1, 9.3±0.1, and 11.4±0.1 degrees.

6. Anhydrous crystalline valaciclovir hydrochloride characterized by substantially the same Raman spectrum as FIG. 3, wherein said Raman spectrum is obtained using a FT-Raman spectrometer at 4 cm⁻¹.

7. Anhydrous crystalline valaciclovir hydrochloride characterized by a Raman spectrum obtained using a FT-Raman spectrometer at 4 cm⁻¹ resolution, wherein said Raman spectrum comprises at least four peaks selected from the group consisting of 1684±1, 1364±1, 1348±1, 1191±1, and 810±1 cm−1.

8. Anhydrous crystalline valaciclovir hydrochloride characterized by substantially the same solid state nuclear magnetic resonance (NMR) spectrum as FIG. 4, wherein said solid state NMR is obtained on a spectrometer operating at a frequency of 90.55 MHz for ¹³C observation at a temperature of 300K, a spinning speed of 10 kHz and a recycle delay of 15 seconds.

9. Anhydrous crystalline valaciclovir hydrochloride characterized by a solid state NMR spectrum obtained using a spectrometer operating at a frequency of 90.55 MHz for ¹³C observation at a temperature of 300K, a spinning speed 10 kHz and a recycle delay of 15 seconds, wherein said solid state NMR comprises chemical shifts at 15.1±0.1, 17.2±0.1, 20.2±0.1, 20.9±0.1, 29.2±0.1, 29.9±0.1, 58.4±0.1, 64.6±0.1, 66.8±0.1, 69.3±0.1, 70.7±0.1, 73.9±0.1, 74.4±0.1, 116.6±0.1, 117.3±0.1, 140.4±0.1, 150.4±0.1, 151.3±0.1, 153.6±0.1, 158.3±0.1, 169.1±0.1 and 169.6±0.1 ppm

10. A pharmaceutical composition comprising the anhydrous crystalline valaciclovir hydrochloride according to claim 3.

11. The pharmaceutical composition according to claim 10 further comprising one or more pharmaceutically acceptable carriers or diluents.

12. A composition comprising the anhydrous crystalline valaciclovir hydrochloride according to claim 3 and hydrated valaciclovir hydrochloride.

13. A composition comprising the anhydrous crystalline valaciclovir hydrochloride according to any of claim 3 and Form 1 valaciclovir hydrochloride.

14. A method for the treatment or prophylaxis of a herpes viral infection in a mammal comprising administering to the mammal, an effective amount of the anhydrous crystalline valaciclovir hydrochloride according to claim 3.

15. The method according to claim 14 wherein said herpes viral infection is selected from the group consisting of herpes simplex virus 1, herpes simplex virus 2, cytomegalovirus, Epstein Barr virus, varicella zoster virus, human herpes virus 6, human herpes virus 7, and human herpes virus 8.

16. A method for the treatment or prophylaxis of a condition or disease associated with a herpes viral infection in a mammal, comprising administering to the mammal an effective amount of anhydrous crystalline valaciclovir hydrochloride according to claim 3.

17-19. (canceled)

20. A process for preparing the anhydrous crystalline valaciclovir hydrochloride according to claim 3 comprising slurrying damp valaciclovir hydrochloride or hydrated valaciclovir hydrochloride in a solvent capable of removing water by azeotropic distillation, under azeotropic distillation conditions.

21. A process for preparing the anhydrous crystalline valaciclovir hydrochloride according to claim 3 comprising the steps of:
a) optionally removing unbound process solvent from damp valaciclovir hydrochloride to provide hydrated valaciclovir hydrochloride;
b) slurrying sand damp valaciclovir hydrochloride or said hydrated valaciclovir hydrochloride in a solvent capable of removing water by azeotropic distillation, under azeotropic distillation conditions to prepare siad anhydrous crystalline valaciclovir hydrochloride; and
c) isolating said anhydrous crystalline valaciclovir hydrochloride.

22. The process according to claim 20, wherein said solvent capable of removing water by azeotropic distillation is selected from the group consisting of C₁₋₆alcohols, ketones, ethers, esters, and mixtures thereof.

23. The process according to claim 22, wherein said solvent is selected from the group consisting of butanol, propanol, toluene, ethyl acetate, butyl acetate, methyl isobutyl ketone and mixtures thereof.

24. The process according to claim 20 further comprising drying said anhydrous crystalline valaciclovir hydrochloride.

25. A process for preparing anhydrous crystalline valaciclovir hydrochloride according to claim 3 comprising the steps of:
a) removing unbound process solvent from damp valaciclovir hydrochloride to provide hydrated valaciclovir hydrochloride;
b) slurrying said hydrated valaciclovir hydrochloride in an anhydrous solvent at a temperature of from about ambient temperature to about the boiling point of said anhydrous solvent for a period of time sufficient to convert said hydrated valaciclovir hydrochloride to said anhydrous crystalline valaciclovir hydrochloride; and
c) isolating said anhydrous crystalline valaciclovir hydrochloride.

26. The process according to claim 25 wherein said anhydrous solvent is selected from the group consisting of water-free IMS, methanol, absolute ethanol, toluene, tetrahydrofuran, MIBK and mixtures thereof.

27. The process according to claim 25 wherein said step of slurrying is carried out at a temperature of from about 50 to about 60° C.

28. The process according to claim 25 further comprising the step of drying said anhydrous crystalline valaciclovir hydrochloride.
